(19) United States
(12) Patent Application Publication
(10) Pub. No.: US 2018/0311228 A1
(43) Pub. Date: Nov. 1, 2018

(54) MEDICAL PRODUCT AND METHOD FOR ELIMINATING SYMPTOMS OF NICOTINE WITHDRAWAL

(71) Applicants: Nicholas A. Havercroft, Scunthorpe (GB); Donald M. Pell, St. Petersburg, FL (US)

(72) Inventors: Nicholas A. Havercroft, Scunthorpe (GB); Donald M. Pell, St. Petersburg, FL (US)

(73) Assignee: BioNebicine, Inc., Clearwater, FL (US)

(21) Appl. No.: 16/025,586

(22) Filed: Jul. 2, 2018

Related U.S. Application Data
(63) Continuation-in-part of application No. 15/234,035, filed on Aug. 11, 2016.

(51) Int. Cl. A61K 31/465; A61K 9/00; A61K 9/50; A61K 47/02; A61K 47/12; A61P 3/04; A61P 25/28; A61P 25/30

(52) U.S. Cl. CPC A61K 31/465; A61K 9/0078; A61K 9/50; A61P 25/30; A61K 47/12; A61P 3/04; A61P 25/28; A61K 47/02

(57) ABSTRACT

In essence, the present invention relates to a medical product containing a normal saline solution and about 1/8^{th} of 1 milligram and 3 milligrams of nicotine. The medical product may also include about 1% to 3% of flavor such as tobacco, menthol and hazelnut. The medical product may also include about 1 to 3 microns that are drawn through an entrainment port, a capsule, the grid and into the individual's lungs and their bloodstream. The present invention is also used for patients who want to maintain the benefits of nicotine such as alertness, memory retention, relaxation, maintaining normal bowel movement, and avoidance of weight gain, without the health risks of tobacco ingredients. It may also be used as a method to initiate weight loss in obese individuals as part of a plan including diet and exercise.

### CROSS REFERENCE TO RELATED APPLICATIONS

The present application is a Continuation-In-Part of U.S. patent application Ser. No. 15/234,035, filed Aug. 11, 2016, and priority is hereby claimed under 35 U.S.C. § 120 based on the earlier application and is hereby incorporated by reference in its entirety into the present application.

### FIELD OF THE INVENTION

This invention relates to a medical product and method for eliminating the symptoms of nicotine withdrawal for those addicted to nicotine as a result of the use of tobacco products. This liquid is disposed in a bar coded or chip regulated glass or plastic capsule that will only work with one of Applicant's dispensers, a micro mesh nebulizer that is programmed to dispense relatively pure nicotine with a 1 to 3 micron particle size.

### BACKGROUND FOR THE INVENTION

It is common for former smokers to state that quitting smoking was the hardest thing that they have ever done. An observation of a physician who worked in an addiction center is that many former addicts continue to smoke, and often comment that overcoming drug addiction was easy compared to quitting smoking. Furthermore, smoking was not illegal, and was relatively cheaper than drugs.

Within about two hours of smoking a cigarette the human body craves another, and with increased frequency the craving becomes stronger. Cigarettes provide almost instant gratification.

In today's market there have been a number of approaches to overcome an addiction to nicotine. Many nicotine replacement products have been unsatisfactory due to delivery of low levels of nicotine. However, one approach as disclosed in our earlier U.S. patent application Ser. No. 15/174,178 filed on Jun. 6, 2016 and entitled Pulmonary Device, Method and Systems for Delivering a Pharmaceutical Product to An Individual has been effective to eliminate the symptoms of withdrawal from a nicotine containing product. Further, the following patent applications were filed by one of the inventors before disclosing such applications to the other inventors. However, the concentration in those applications were far in excess for a safe delivery by a micro mesh nebulizer.

As disclosed in our aforementioned patent application it has now been recognized that inhaling very small amounts of 1 to 3 micron particles of a pure water nicotine solution or a nicotine saline solution will avoid withdrawal symptoms. Nevertheless, it is important to prevent the inhaler from delivering an excess amount of nicotine particularly for young persons and the potential death of pets due to the ingestion of one or more capsules. Accordingly, an inhaler for limiting the time of an initial inspiration and to prevent second or subsequent inspirations for a preselected time following an initial or previous inspiration is provided. It is also important to limit the inhaler to use replaceable cartridges as opposed to refillable containers to avoid having relatively untrained individuals working with toxic materials and exposing the nicotine solution to contamination, bacteria, fungus and yeast.

It is well known that some young people start smoking to lose weight. Therefore, it is reasonable to assume that Applicant's delivery device using incrementally increasing doses of a nicotine solution starting at ⅛ milligram and increasing as tolerated to 1 or 1 ½ milligrams of nicotine solution could accomplish significant weight loss as it is well known and accepted that nicotine increases the metabolic rate by 10 percent, and suppresses appetite. Therefore, a 150 pound person using a decreasing amount of nicotine could lose 15 pounds of weight as a starting point for further weight loss.

Notwithstanding the above, it is presently believed that there is a need and a potential commercial market for a medical product and method in accordance with the present invention.

### SUMMARY OF THE INVENTION

A threaded glass capsule containing a Radio Frequency Identification (RFID) encryption sprayed on the capsule and a threaded open end containing a removable silicone cap is provided. The code contains information regarding the drug, concentration, and expiration date. Once the cap is removed and the capsule is securely threaded into the micro micron mesh nebulizer, the device recognizes the legitimacy of its contents and will function. The bar code prevents reuse of the capsule to avoid contamination, dosing errors and adulteration of its contents. The encryption also allows the nebulizer to signal the payor, the prescriber, and the pharmacy using blue tooth technology and the individuals' smart phone to confirm user compliance. HIPAA compliance regulations will be observed. The encryption also avoids injury to the grid by debris and contaminants The glass capsules are produced in a mold and are 37 mm tall and 14 mm wide with an open external end of 11 mm They are intended solely for use in the patent pending micro micron mesh nebulizer.

Threaded glass capsules in various sizes, shapes, compositions and colors all for various applications are available from a number of U.S. or foreign manufacturers as for example Richland Glass of Vineland, N.J. in the U.S.

In essence, the present invention contemplates a medical product and a method for eliminating the symptoms of withdrawal from nicotine containing products such as cigarettes or other tobacco products. This medical product contains pure water and relatively pure nicotine or a mixture of 0.9% sodium chloride in water mixed with nicotine.

In the present invention, the Applicant's effort is to provide nicotine in the pattern and concentration to which those addicted to nicotine are accustomed.

In a preferred embodiment of the invention, a medical product is enclosed in a bar coded or chip regulated capsule that includes a specific dose of nicotine and may also include 1% citric acid for taste and up to 3% flavoring to mask the bitterness of nicotine.

As stated above in paragraph [0007], it is well known that some young people start smoking to lose weight. Therefore, it is reasonable to assume that Applicant's delivery device and composition in using incrementally increasing doses of a nicotine solution starting at ⅛ milligram and increasing as tolerated to 1 or 1 ½ milligrams of nicotine solution could accomplish significant weight loss as it is well known and accepted that nicotine increases the metabolic rate by 10 percent, and suppresses appetite. Therefore, a 150 pound person using a decreasing amount of nicotine could lose 15 pounds of weight as a starting point for further weight loss.

A second embodiment of the invention relates to a method for eliminating the symptoms of withdrawal from a nicotine product by adding small concentrations of nicotine to the above solution. The amount of nicotine in a capsule containing 1 to 3 milliliters of mixture (solution) contains from about 1/8^{th} of a milligram to 3 milligrams of nicotine in decreasing amounts.

### DESCRIPTION OF THE PREFERRED EMBODIMENTS OF THE INVENTION

In a preferred embodiment of the present invention a pure water nicotine solution or a nicotine/saline solution contains nicotine dissolved in a 0.9% sodium chloride solution with 1% citric acid for taste and up to about 3% of a flavor.

A formulation method for a nicotine solution dissolved in water or in a 0.9% Sodium chloride solution with nicotine and with additives to be used with a micro micron mesh ultrasonic nebulizer to eliminate withdrawal symptoms in those addicted to nicotine. While Applicant's have referred to a micron micro mesh nebulizer, it should be recognized that any device that will produce particle sizes of 1-3 microns of saline/nicotine mixture or solution as defined herein may be substituted for the micron micro mesh nebulizer. However, no patent pending bar coded lockout capsule will work in Applicants' patent pending nebulizer unless they are designed and/or programmed to do so. This assures accurate dosing. It should be recognized that the capsule can be modified for other pharmaceuticals and specific doses thereof such that the pharmaceuticals in the future are provided in a specific dose or in the future a more sophisticated capsule or cartridge can be used to select different doses.

This application describes a formula for use with a micro micron mesh nebulizer in order to deliver solutions containing low concentrations of nicotine into the lungs and almost immediately into the bloodstream and the patient's brain in 1-3 micron particle sizes. The mixture solution is used to prevent withdrawal symptoms in those addicted to nicotine as a result of their use of tobacco products.

The necessity of eliminating withdrawal symptoms is important in a successful attempt to eliminate their use of tobacco products. It is the belief of the applicants that if equals are substituted for equals abstinence should occur. This embodiment describes the use of disposable capsules or cartridges to which are added small amounts of nicotine as well as up to 3% flavoring, and 1% citric acid for taste. These concentrations may vary. These solutions are contained in a capsule or cartridge made of glass, and perhaps plastic or silicone in those countries where it would be permissible. The volume of these is preferably 1-3 milliliters. These containers or capsules are threaded and have a silicone cap. Furthermore, the capsule is threaded and can be secured tightly in a nebulizing device.

When the cartridge is placed in the nebulizer cylinder anterior to the micro micron mesh grid; the cap is removed. The grid is activated by an on off switch and the solution is pulled through the device by the user drawing air through an entrainment port, the cylinder, the cartridge and the grid. The content of the nicotine solution varies in a declining manner in each cartridge from three milligrams, tapering by one eight of a milligram in a stepwise fashion. The lowest amount of nicotine in a cartridge is one eight of a milligram. The amount of nicotine described in this application may be modified depending on the severity of the nicotine addiction, but is not expected to exceed 3 milligrams per cartridge. To mask to taste of nicotine numerous flavors are available including citric acid, tobacco, apple, cherry, maple, menthol, just to name a few.

This allows an addicted individual to taper down the amount of nicotine needed to avoid withdrawal symptoms and hopefully quit the addiction completely and therefore eliminate the use of tobacco products.

The delivery into the lungs and therefore into the bloodstream of this solution and its contents is extremely efficient allowing 90% absorption rapidly in a pattern and concentration to which the nicotine addicted individual is accustomed. No heating of any liquid is involved. No refillable cartridge or capsule will be used thus avoiding dosage errors, spillage and contamination. The small doses of nicotine involved will avoid toxicity if a child or pet accidentally ingests a cartridge. No aerosol effluent is produced as the solution is housed in a cartridge and is drawn into the user's lungs by inhalation. The grid has multiple perforations of various sizes shapes, and number and is electrified by two 1.5 volt rechargeable lithium titanate batteries. This produces two micron sized particles. The sole source of flow through the device is the user's inspiratory effort and allows a rapid blood level of nicotine to be attained in a pattern to which nicotine addicted individuals are accustomed.

The pH of these solutions ranges from 5.5 to 7.4 for comfort and to decrease airway irritation and coughing. A pH of 7.4 is preferred as it enhances delivery of nicotine into the bloodstream.

This patent pending glass capsule works only with Applicants' patent pending nebulizer guaranteeing a one-way flow to the user when the grid is activated. The capsule and/or nebulizer provides an initial four second activation period followed by a lockout period. This lockout period of about 15 seconds allows the user to exhale when the grid is not activated to avoid wasted medication. This device can also act as a safe source of nicotine for those who cannot or will not give up a dependence on nicotine and who want the benefits of nicotine without the inherent risks of tobacco products. The benefits include mental alertness, improved memory retention, relaxation, improved bowel function and avoidance of weight gain.

In a preferred or first embodiment of the invention, the medical product for eliminating the symptoms of nicotine withdrawal from a nicotine containing product includes a mixture of a saline solution containing between ⅛^{th} and 3 milligrams of nicotine, 1% citric acid for taste and 1% to 3% of a flavor, and wherein the medical product has an alkaline pH of about 7.4.

In a second embodiment of the invention, the medical product for eliminating the symptoms of withdrawal from a mixture containing product according to paragraph [0023], in which the saline solution is a 0.9% sodium chloride solution in water and in which the flavors are selected from the group consisting of citrus, tobacco, apple, cherry, maple, menthol, hazelnut, peach, lemon, vanilla and chocolate.

In a third embodiment of the invention, the medical product for eliminating the symptoms of withdrawal from a mixture containing product according to paragraph [0024], which includes a capsule containing 3 to 5 ml of said mixture disposed in said capsule and wherein each capsule contains between about ⅛ milligram and 3 milligrams of nicotine and includes a specific amount of nicotine.

In a fourth embodiment of the invention, the medical product for eliminating the symptoms of withdrawal from a mixture containing product according to paragraph [0024], in which each capsule contains about ¼ milligram of nicotine and wherein the nebulizer is programmable with variations of timing for each breath and/or changes in frequency of the micro mesh grid.

A still further embodiment of the invention in which the medical product for eliminating the symptoms of withdrawal from a mixture containing product according to paragraph [0026], in which each capsule contains about ⅝ milligram of nicotine.

A still further embodiment of the invention in which the medical product for eliminating the symptoms of withdrawal from a mixture containing product according to paragraph [0027], in which each capsule contains about ¾ milligram of nicotine.

A still further embodiment of the invention in which the medical product for eliminating the symptoms of withdrawal from a mixture containing product according to paragraph [0028], in which each capsule contains about ⅞ milligram of nicotine.

A further embodiment of the invention in which the medical product for eliminating the symptoms of withdrawal from a mixture containing product according to paragraph [0029], in which each capsule contains about 1 milligram of nicotine.

A still further embodiment of the invention in which the medical product for eliminating the symptoms of withdrawal from a mixture containing product according to paragraph [0030], in which each capsule contains about 1 ¼ milligrams of nicotine.

A still further embodiment of the invention in which the medical product for eliminating the symptoms of withdrawal from a mixture containing product according to paragraph [0031], in which each capsule contains about 1 ⅜ milligrams of nicotine.

A still further embodiment of the invention in which the medical product for eliminating the symptoms of withdrawal from a mixture containing product according to paragraph [0032], in which each capsule contains about 1 ½ milligrams of nicotine.

A still further embodiment of the invention in which the medical product for eliminating the symptoms of withdrawal from a mixture containing product according to paragraph [0033], in which each capsule contains about 1 ⅝ milligrams of nicotine.

A still further embodiment of the invention in which the medical product for eliminating the symptoms of withdrawal from a mixture containing product according to paragraph [0034], in which each capsule contains about 1 ¾ milligrams of nicotine.

A still further embodiment of the invention in which the medical product for eliminating the symptoms of withdrawal from a mixture containing product according to paragraph [0035], in which each capsule contains about 2 milligrams of nicotine.

A still further embodiment of the invention in which the medical product for eliminating the symptoms of withdrawal from a mixture containing product according to paragraph [0036], in which each capsule contains about 2 ¼ milligrams of nicotine.

A still further embodiment of the invention in which the medical product for eliminating the symptoms of withdrawal from a mixture containing product according to paragraph [0037], in which each capsule contains about 2 ½ milligrams of nicotine.

A still further embodiment of the invention in which the medical product for eliminating the symptoms of withdrawal from a mixture containing product according to paragraph [0038], in which each capsule contains about 2 ¾ milligrams of nicotine.

A still further embodiment of the invention in which the medical product for eliminating the symptoms of withdrawal from a mixture containing product according to paragraph [0039], in which each capsule contains about 3 milligrams of nicotine.

A still further embodiment of the invention in which the method for eliminating the symptoms of withdrawal from a nicotine containing product, the method comprising:

a. providing a saline/nicotine solution containing from about one (1) to about three (3) milligrams of nicotine saline solution;

b. nebulizing said saline/nicotine solution with an electrically charged micro mesh grid to produce a nicotine particle size of between about 1 and about 3 microns;

c. inhaling the 1 to 3 micron particles of a nebulized nicotine concentration for a first preselected period of time;

d. providing a second reduced concentration of a saline/nicotine solution;

e. nebulizing said second reduced concentration with a micro mesh grid to produce nicotine particles of about 2 microns; and

f. inhaling the 2 micron particles from step e for a second period of time.

A still further embodiment of the invention in which the method for eliminating the symptoms of withdrawal from inhaling a nicotine containing product according to paragraph [0041], in which the micro micron mesh grid is electrified to vibrate the grid in step b and e by two 1.5 volt lithium titanate batteries; and in which the step e and f are each repeated until the individual has eliminated the symptoms of withdrawal.

While the invention has been described in connection with its preferred embodiments it should be recognized that changes and modifications may be made therein without departing from the scope of the appended claims.

What is claimed is:

1. A medical product for eliminating the symptoms of nicotine withdrawal from a nicotine containing product, said medical product comprising:
a mixture of water and/or a saline solution each containing between ⅛th and 3 milligrams of nicotine and 1% citric acid for taste; and
wherein said medical product has an alkaline pH of about 7.4; and, in which said product contains a single specific product with a specific amount of nicotine deliverable in multiple doses of the specific amount of nicotine.

2. The medical product for eliminating the symptoms of withdrawal from a mixture containing product according to claim 1, in which the saline solution is a 0.9% sodium chloride solution in water and in which said flavors are selected from the group consisting of citrus, tobacco, apple, cherry, maple, menthol, hazelnut, peach, lemon, vanilla and chocolate.

3. The medical product for eliminating the symptoms of withdrawal from a mixture containing product according to claim 2, which includes a capsule containing 3 to 5 ml of said mixture disposed in said capsule and wherein each capsule contains between about ⅛ milligram and 3 milligrams of nicotine and includes a specific amount of nicotine.

4. The medical product for eliminating the symptoms of withdrawal from a mixture containing product according to claim 1, in which each capsule contains about ¼ milligram of nicotine and wherein the nebulizer is programmable with variations of timing for each breath and/or changes in frequency of the micro mesh grid.

5. The medical product for eliminating the symptoms of withdrawal from a mixture containing product according to claim 1, in which each capsule contains about ⅝ milligram of nicotine.

6. The medical product for eliminating the symptoms of withdrawal from a mixture containing product according to claim 1, in which each capsule contains about ¾ milligram of nicotine.

7. The medical product for eliminating the symptoms of withdrawal from a mixture containing product according to claim 1, in which each capsule contains about ⅞ milligram of nicotine.

8. The medical product for eliminating the symptoms of withdrawal from a mixture containing product according to claim 1, in which each capsule contains about 1 milligram of nicotine.

9. The medical product for eliminating the symptoms of withdrawal from a mixture containing product according to claim 1, in which each capsule contains about 1 ¼ milligrams of nicotine.

10. The medical product for eliminating the symptoms of withdrawal from a mixture containing product according to claim 1, in which each capsule contains about 1 ⅜ milligrams of nicotine.

11. The medical product for eliminating the symptoms of withdrawal from a mixture containing product according to claim 1, in which each capsule contains about 1 ½ milligrams of nicotine.

12. The medical product for eliminating the symptoms of withdrawal from a mixture containing product according to claim 1, in which each capsule contains about 1 ⅝ milligrams of nicotine.

13. The medical product for eliminating the symptoms of withdrawal from a mixture containing product according to claim 1, in which each capsule contains about 1 ¾ milligrams of nicotine.

14. The medical product for eliminating the symptoms of withdrawal from a mixture containing product according to claim 1, in which each capsule contains about 2 milligrams of nicotine.

15. The medical product for eliminating the symptoms of withdrawal from a mixture containing product according to claim 1, in which each capsule contains about 2 ¼ milligrams of nicotine.

16. The medical product for eliminating the symptoms of withdrawal from a mixture containing product according to claim 1, in which each capsule contains about 2 1/2 milligrams of nicotine.

17. The medical product for eliminating the symptoms of withdrawal from a mixture containing product according to claim 1, in which each capsule contains about 2 ¾ milligrams of nicotine.

18. The medical product for eliminating the symptoms of withdrawal from a mixture containing product according to claim 1, in which each capsule contains about 3 milligrams of nicotine.

19. A method for eliminating the symptoms of withdrawal from a nicotine containing product, said method comprising:
a. providing a saline/nicotine solution containing from about one (1) to about three (3) milligrams of nicotine saline solution;
b. nebulizing said saline/nicotine solution with an electrically charged micro mesh grid to produce a nicotine particle size of between about 1 and about 3 microns;
c. inhaling the 1 to 3 micron particles of a nebulized nicotine concentration for a first preselected period of time;
d. providing a second reduced concentration of a saline/nicotine solution;
e. nebulizing said second reduced concentration with a micro mesh grid to produce nicotine particles of about 2 microns; and
f. inhaling the 2 micron particles from step e for a second period of time.

20. The method for eliminating the symptoms of withdrawal from inhaling a nicotine containing product according to claim 19, in which the micro micron mesh grid is electrified to vibrate said grid in step b and e by two 1.5 volt lithium titanate batteries; and
in which the step e and f are each repeated until the individual has eliminated the symptoms of withdrawal.

21. A method for initiating weight loss in those individuals using incremental doses of nicotine starting at ⅛ of a milligram of nicotine per treatment and increasing to 1 to 1 ½ milligrams of nicotine as tolerated allowing the patient to lose 10 percent of body mass by increasing the metabolic rate and suppressing the appetite.

22. A medical product for the delivery of nicotine for those who cannot or will not give up their addiction to nicotine because of the benefits including mental alertness, improved memory retention, relaxation, improved bowel function and the avoidance of weight gain without the inherent risks of other tobacco ingredients.

23. A threaded glass capsule containing a Radio Frequency Identification (RFID) encryption is used to dispense a pharmaceutical liquid.

24. A threaded glass capsule open on one end and sealed by silicone cap for containing a pharmaceutical liquid.

25. A glass capsule threaded on one end and containing a bar code sprayed on it which signals the micro micron mesh nebulizer it is safe to function.

26. A glass capsule threaded on one end contains a barcode which prevents reuse of said capsule to avoid contamination, dosage errors, and adulteration.

27. A glass capsule threaded on an open end and containing a barcoded message which signals the micro micron mesh nebulizer of its authenticity and allows the nebulizer to send a signal to the individual's smart phone allowing it to inform the payor, the prescriber, and the pharmacy to prove user compliance.

28. A glass capsule threaded on one end and open on that end that is 37 mm tall, 17 mm wide and 11 mm on the open end.

29. A glass threaded capsule open on the threaded end for a single use only to prevent potential damage to the nebulizer by contaminants and debris.

30. A glass threaded capsule open on the threaded end for a multiple use only of a single treatment.
